(19) United States
(12) Patent Application Publication
(10) Pub. No.: US 2026/0108649 A1
(43) Pub. Date: Apr. 23, 2026

(54) Ammonia Control for Animals Reared in Confinement

(71) Applicants: Michael Putnam, Nacogdoches, TX (US); Joey Bray, Nacogdoches, TX (US); Anton Ward, Sarcoxie, MO (US)

(72) Inventors: Michael Putnam, Nacogdoches, TX (US); Joey Bray, Nacogdoches, TX (US); Anton Ward, Sarcoxie, MO (US)

(21) Appl. No.: 19/366,657

(22) Filed: Oct. 23, 2025

Related U.S. Application Data
(60) Provisional application No. 63/710,922, filed on Oct. 23, 2024.

(51) Int. Cl. A61L 9/012

(52) U.S. Cl. CPC A61L 9/012

(57) ABSTRACT

Methods of controlling ammonia in an animal containment structure having at least one consumption area and at least one spatially distinct bedding area are described. A method includes maintaining at least one animal in the animal containment structure for a maintenance period; dispersing a first conditioner onto the consumption apron at a first frequency during the maintenance period; and dispersing a second conditioner onto bedding pack in the bedding area at a second frequency during the maintenance period, the second frequency being less than the first frequency. Animal containment structures are also described.

### FIELD

The disclosure relates generally to the field of livestock management. More particularly, the disclosure relates to methods of controlling ammonia in an animal containment structure. Specific examples described herein relate to methods of controlling ammonia in cattle production structures.

### BACKGROUND

As animal waste builds in animal containment structures, bacteria can proliferate on organic bedding and other surfaces. Ammonia levels can rise quickly and significantly, which can create an undesirable and potentially hazardous environment for both animals and humans. The pungent odor of ammonia is a common indicator of its presence, and prolonged exposure can irritate respiratory systems, reduce animal growth rates, and compromise overall health.

Conditioners have been applied to animal bedding in efforts to suppress the bacterial proliferation that ultimately results in ammonia buildup. To date, however, conventional application procedures have suggested a limited beneficial effect of the use of such conditioners. For example, Hogan et al. describe an evaluation of the persistence of the antimicrobial benefits of applying a conditioner comprising 93% sodium bisulfate to bedding in free stalls. (Bacterial Counts in Organic Materials Used as Free-Stall Bedding Following Treatment with a Commercial Conditioner, J. Dairy Sci. 90:1058-1062 (2007)). In that study, the conditioner was spread evenly over fresh bedding following stall cleaning. While application of the conditioner resulted in an immediate reduction in bacterial counts, the effect was short-lived, with treated bedding showing no difference in bacterial counts relative to control bedding as quickly as two days after application of the conditioner.

The fleeting effects of application of a conditioner to only the bedding in an animal containment structure has limited the use of these conditioners in animal production.

A need exists, therefore, for new and improved methods of controlling ammonia in an animal containment structure, such as cattle production structures, and improved animal containment structures.

### BRIEF SUMMARY OF SELECTED EXAMPLES

Various example methods of controlling ammonia in an animal containment structure are described herein.

An example method of controlling ammonia in an animal containment structure comprises maintaining at least one animal in the animal containment structure for a maintenance period of at least two weeks; and dispersing a conditioner onto the consumption apron once each week during the maintenance period. In specific examples, the conditioner is an acidic conditioner, such as sodium bisulfate.

Another example method of controlling ammonia in an animal containment structure comprises maintaining at least one animal in the animal containment structure for a maintenance period of at least two weeks; dispersing a first conditioner onto the consumption apron; dispersing a second conditioner onto the bedding pack; and at least one week after performing the dispersing a first conditioner onto the consumption apron and after performing the dispersing a second conditioner onto the bedding pack, dispersing a third conditioner onto the consumption apron. In specific examples, each of the first, second, and third conditioners is an acidic conditioner, such as sodium bisulfate.

Another example method of controlling ammonia in an animal containment structure comprises maintaining at least one animal in the animal containment structure for a maintenance period of at least eight weeks; dispersing a first acidic conditioner comprising sodium bisulfate over the at least one animal and onto the consumption apron once each week during the maintenance period; and dispersing a second acidic conditioner comprising sodium bisulfate over the at least one animal and onto the bedding pack.

In some example methods, one, more than one, or all steps of dispersing a conditioner are performed from a position within the animal containment structure.

In some example methods, one, more than one, or all steps of dispersing a conditioner are performed while at least one animal is positioned within the animal containment structure.

In some example methods, one, more than one, or all steps of dispersing a conditioner comprise dispersing a conditioner over at least one animal.

In some example methods, all conditioners used in steps of dispersing a conditioner comprise the same active ingredient.

In some example methods, all conditioners used in steps of dispersing a conditioner comprise dispersing an acidic conditioner.

In some example methods, all conditioners used in steps of dispersing a conditioner comprise sodium bisulfate.

Various example animal containment structures suitable for use in example methods are also described.

An example animal containment structure comprises an outer perimeter that defines an inner containment area that includes at least one consumption area having a consumption apron and at least one bedding area that is spatially distinct from the at least one consumption area, the bedding area separated from the consumption area by a working area; and a spreader disposed in the working area, the spreader including a hopper and a conditioner, such as an acidic conditioner comprising sodium bisulfate as the active ingredient, disposed in the hopper, the spreader configured to disperse the conditioner in according with dispersing steps of methods according to embodiments.

Additional understanding of these example methods of controlling ammonia in an animal containment structure can be obtained by review of the detailed description of selected examples, below, and the referenced drawings.

### BRIEF DESCRIPTION OF FIGURES

FIG. 1 is a schematic illustration of an animal containment structure.

FIG. 2 is a flowchart illustration of an example method of controlling ammonia in an animal containment structure.

FIG. 3 is a flowchart illustration of another example method of controlling ammonia in an animal containment structure.

FIG. 4 is a flowchart illustration of another example method of controlling ammonia in an animal containment structure.

### DETAILED DESCRIPTION OF SELECTED EXAMPLES

The following detailed description and the appended drawings describe and illustrate various example methods of controlling ammonia in an animal containment structure. The description and illustration of these examples are provided to enable one skilled in the art to perform example methods of controlling ammonia in an animal containment structure. The inclusion of detailed descriptions of these examples is not intended to limit the scope of the invention, or its protection, in any manner. The invention is capable of being practiced or carried out in various ways and the examples described and illustrated herein are not considered exhaustive.

As used herein, the term “conditioner” refers to a composition intended to lower the pH of liquids produced during or derived from agricultural operations, such as liquids obtained from an animal containment structure and solutions prepared by mixing solid matter obtained from an animal containment structure with a suspension fluid, diluent, or solvent. Examples of conditioners include, but are not limited to, acidic conditioners, such as sodium bisulfate, lime, adsorbers, compositions that inhibit the conversion of urea to ammonia, and biological agents, such as bacteria, enzymes, and oils that inactivate ammonia.

As used herein, the term “active ingredient” refers to the specific compound within a composition that is primarily responsible for achieving the intended effect of using the composition for its intended purpose.

As used herein, the term “mixture” refers to a composition including two or more materials. The materials can be distributed within the composition in any suitable manner, including within fully separate layers, homogenously distributed throughout the composition, and any distribution of the materials between these two distributions.

The inventor has surprisingly discovered that dispersing a conditioner, such as the acidic conditioner sodium bisulfate, onto a consumption apron in an animal containment structure can provide beneficial ammonia control. The inventor has also surprisingly discovered that dispersing a conditioner, such as the acidic conditioner sodium bisulfate, onto a consumption apron and onto bedding pack in an animal containment structure, at different frequencies, can provide beneficial ammonia control and avoid the need for daily application of a conditioner to bedding pack, eliminating unnecessary time and materials in ammonia control efforts.

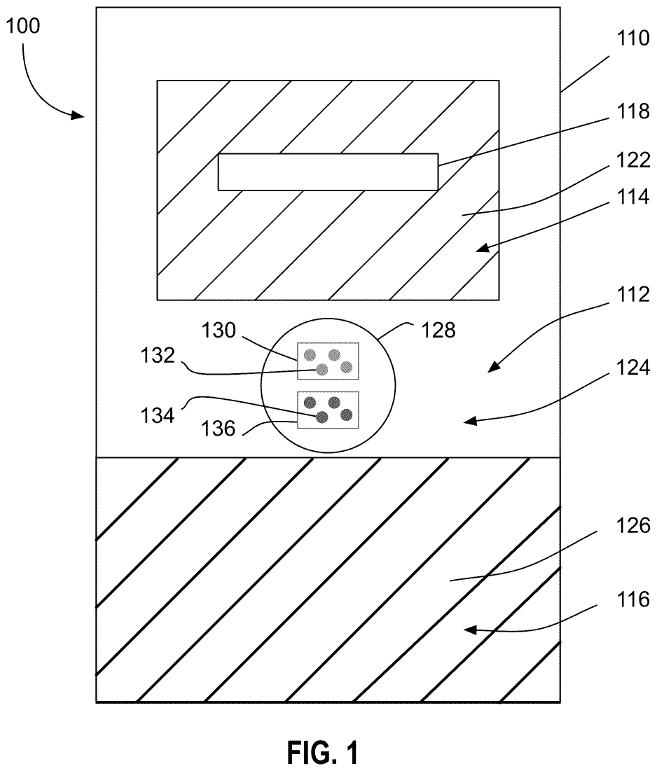

FIG. 1 illustrates a schematic of an animal containment structure 100 that can be used in the performance of methods according to embodiments. The animal containment structure 100 is schematic in nature; dimensions, proportions, and layout of the animal containment structure 100 are intended solely to provide a reference for the example methods described herein. The inventive methods are capable of being performed in association with animal containment structures having a variety of dimensions, proportions, and layouts.

The animal containment structure 100 has an outer perimeter 110 that defines an inner containment area 112. In the illustrated example, outer perimeter 110 is a series of connected side walls, one or more of which may define one or more openings to provide ventilation or a passageway through which a conditioner can be dispersed from the outside of the animal containment structure 110 into the inner containment area 112. The inner containment area 112 includes at least one consumption area 114 and at least one bedding area 116. The consumption area 114 includes a consumption bunk 118 into which food, water, or both, can be disposed for consumption by one or more animals contained within the animal containment structure 100. Example structures for the consumption bunk include, but are not limited to, troughs, tubs, buckets, and other suitable structures. The consumption bunk 118 is disposed on a consumption apron 122, which comprises a rigid, artificial structure that can be cleaned by mechanical scraping, spraying with a fluid, or other process. Example structures for a consumption apron 122 include, but are not limited to, grates, concrete slabs, and other suitable structures. The bedding area 116 is spatially distinct from the consumption apron 122 and, in the illustrated example animal containment structure 100, from the consumption area 114. In the illustrated example, the bedding area 116 is spaced from consumption area 114 by a working area 124, which can comprise open space within the animal containment area 100, one or more alleys, one or more chutes, or other suitable structure or space. In the illustrated example animal containment structure 100, bedding pack 126 is disposed on the ground in the bedding area 116, providing a suitable space for the one or more animals contained within the animal containment area 100 to rest.

In the illustrated example, a spreader 128 is disposed in the working area 124 and includes hopper 130. A conditioner 132, such as an acidic conditioner comprising sodium bisulfate as the active ingredient, is disposed in the hopper 130. The spreader 128 is configured to disperse the conditioner in according with dispersing steps of methods according to embodiments. For example, the spreader 128 can be configured to disperse a conditioner onto the consumption apron 122. In this example, the spreader 128 can be configured to not disperse the conditioner onto the bedding area 116. In other examples, the spreader 128 is configured to disperse a first conditioner 132 onto the consumption apron 122 and a second conditioner 134 on the bedding area 116. In these examples, each of the first 132 and second 134 conditioners can comprise an acidic conditioner and can comprise the same active ingredient, such as sodium bisulfate, or the first 132 and second 134 conditioners can comprise different active ingredients. Also in these examples, the spreader 128 can include only a single hopper 130 or first 130 and second 136 hoppers. In these examples, the first conditioner 132 can be disposed in one of the two hoppers 130, 136 and the second acidic 134 conditioner can be disposed in the second hopper 136.

The spreader 128 can include any suitable structure for dispersing conditioner contained within the one or more hoppers 130, 136. For example, the spreader can include a rotatable dispersion member that throws conditioner falling onto it through gravity action on conditioner exiting the one or more hoppers 130, 136 through an exit opening in the one or more hoppers 130, 136. Also, the spreader 128 can be adapted for dispersing conditioner over at least one animal contained within the animal containment structure 100. For example, the one or more hoppers 132, 134 and the dispersion member or other structure for dispersing conditioner can be disposed above the ground and above expected heights of the one or more animals intended to be contained within the animal containment structure 100. In some examples, the spreader 128 includes or is disposed on a frame disposed on the ground, elevating the one or more hoppers 132, 134 and the dispersion member or other structure for dispersing conditioner to a suitable height. In other examples, the spreader is attached to an upper closure on the animal containment structure 100, such as a trusses or other structural members disposed above the at least one consumption area 114 and one bedding area 116. In other examples, the spreader 128 is external to the animal containment structure 100. In these examples, the animal containment structure 100 defines an opening, such as a window or open side wall, through which the spreader 128 can disperse the conditioner. For example, a spreader 128 can be disposed outside of the animal containment structure 100, adjacent to an elevated opening in a side wall of the animal containment structure, with a dispersion member or other structure for dispersing conditioner disposed adjacent the elevated opening in the side wall so the dispersion member or other structure for dispersing conditioner can disperse the conditioner through the elevated opening in the sidewall of the animal containment structure and onto the at least one consumption area 114 and at least one bedding area 116 of the animal containment structure, as appropriate.

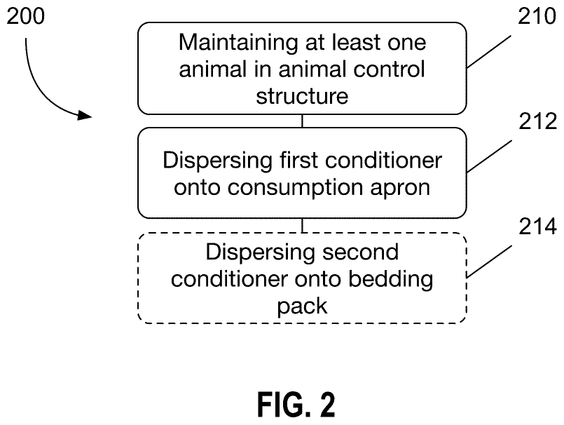

FIG. 2 is a flowchart illustration of an example method 200 of controlling ammonia in an animal containment structure having at least one consumption apron and at least one bedding area having bedding pack disposed therein. Performance of the method 200 results in controlling ammonia in the animal containment structure by lowering, at least temporarily, the pH of one or more of liquids used or produced during or derived from agricultural operations within the animal containment structure, such as liquid animal waste, and organic solids used or produced during or derived from agricultural operations within the animal containment structure, such as bedding and solid animal waste, such as manure. For example, performance of the method 200 results in lowering, at least temporarily, the pH of a liquid obtained from the surface of the consumption apron of the animal containment structure with which the method 200 is performed, such as liquid animal waste or a solution containing liquid animal waste obtained from the surface of the consumption apron of the animal containment structure. Also, performance of the method 200 can result in lowering, at least temporarily, the pH of a liquid solution prepared by mixing solid matter obtained from an animal containment structure, such as bedding or other solid matter, with a suspension fluid, such as sterile distilled water. The method 200 can be performed with any suitable animal containment structure having at least one consumption area having a consumption apron and at least one bedding area that is spatially distinct from the at least one consumption area, such as example animal containment structure 100 described above and illustrated in FIG. 1.

An initial step 210 comprises maintaining at least one animal in the animal containment structure for a maintenance period of at least two weeks. This step can be performed by conventional maintenance procedures for the relevant animal being maintained within the animal containment structure and should include at least feeding the at least one animal, cleaning both the at least one consumption apron and at least one bedding area, and replacing any bedding pack contained within the at least one bedding area.

Step 210 can comprise maintaining any suitable animal for commercial agricultural purposes. The at least one animal can be any animal conventionally reared in confinement, including ruminant and non-ruminant animals. Specific examples of suitable ruminant animals include, but are not limited to, cattle, including beef cattle, dairy cattle, and dual-purpose cattle, sheep, and goats. Specific examples of suitable non-ruminant animals included, but are not limited to, poultry, including broiler chickens, layer chickens, dual-purpose chickens, and turkeys, swine, equine, companion animals, rabbits, and reptiles.

Step 210 can be performed to maintain any suitable number of animals, including at least one animal, one animal, two animals, a plurality of animals, three animals, more than three animals, ten animals, twenty animals, thirty animals, forty animals, fifty animals, one hundred animals, more than one hundred animals, one thousand animals, and more than one thousand animals.

Step 210 must be performed for at least two weeks and can be performed for any duration of time beyond two weeks. A skilled artisan will be able to select a suitable duration of time based on various considerations, including, but not limited to, the type, number, and purpose of the at least one animal being maintained in performance of step 210. In one example, step 210 is performed to maintain beef cattle in a finishing phase of production, such as in a feedlot. In this example, step 210 can be performed for twelve to twenty-four weeks. In another example in which step 210 is performed to maintain beef cattle in a finishing phase of production, step 210 is performed for fourteen to twenty weeks. In another example in which step 210 is performed to maintain beef cattle in a finishing phase of production, step 210 is performed for sixteen to eighteen weeks.

Another step 212 comprises dispersing a conditioner onto the consumption apron at a frequency of once each week during the maintenance period. Step 212 can be performed using any suitable conditioner and any suitable process, including use of a spreader as described above with respect to the example animal containment structure 100. Also, step 212 can be performed to disperse the conditioner at any suitable dispersion rate and a skilled artisan will be able to select an appropriate dispersion rate for the conditioner based on various considerations, including the type, number, and purpose of the at least one animal being maintained in performance of step 210, the waste production of the at least one animal being maintained in performance of step 210, the presence of ventilation equipment in the animal containment structure, the absence of ventilation equipment in the animal containment structure, the capacity of ventilation equipment in the animal containment structure, and the age of ventilation equipment in the animal containment structure. The inventors have determined that, in an example in which step 210 is performed to maintain beef cattle in a finishing phase of production, performing step 212 at a dispersion rate of between 75 pounds and 750 pounds of the acidic conditioner sodium bisulfate per 1000 ft² of the consumption apron is suitable. In another example in which step 210 is performed to maintain beef cattle in a finishing phase of production, performing step 212 at a dispersion rate of between 75 pounds and 500 pounds of the acidic conditioner sodium bisulfate per 1000 ft² of the consumption apron is suitable. In another example in which step 210 is performed to maintain beef cattle in a finishing phase of production, performing step 212 at a dispersion rate of between 75 pounds and 250 pounds of the acidic conditioner sodium bisulfate per 1000 ft² of the consumption apron is suitable. In another example in which step 210 is performed to maintain beef cattle in a finishing phase of production, performing step 212 at a dispersion rate of between 75 pounds and 125 pounds of the acidic conditioner sodium bisulfate per 1000 ft² of the consumption apron is suitable.

Step 212 can be performed from a position within the animal containment structure and directly to the consumption apron.

Also, step 212 can be performed while the at least one animal is positioned within the animal containment structure or while the at least one animal is temporarily outside of the animal containment structure. Advantageously, step 212 can be performed such that the conditioner is dispersed over the at least one animal while the at least one animal is in the animal containment structure.

An optional step 214 comprises dispersing a second conditioner onto bedding pack disposed in the bedding area of the animal containment structure. This optional step is performed at a frequency of once every two weeks during the maintenance period of step 210. Thus, at this once every two weeks frequency, optional step 214, if included, is performed at least once since step 210 is performed for at least two weeks. In specific examples, step 214 is performed twice in a method in which step 210 is performed for four weeks, and is performed eight times in a method in which step 210 is performed for sixteen weeks.

If included, step 214 can be performed using any suitable conditioner and any suitable process, including use of a spreader as described above with respect to the example animal containment structure 100. Also, step 214 can be performed to disperse the conditioner at any suitable dispersion rate and a skilled artisan will be able to select an appropriate rate of dispersion for the conditioner based on various considerations, including the type, number, and purpose of the at least one animal being maintained in performance of step 210, the waste production of the at least one animal being maintained in performance of step 210, the presence of ventilation equipment in the animal containment structure, the absence of ventilation equipment in the animal containment structure, the capacity of ventilation equipment in the animal containment structure, and the age of ventilation equipment in the animal containment structure. Step 214 can be performed at the same or different dispersion rate as step 212. For example, in methods using the acidic conditioner sodium bisulfate, both steps 212 and 214 can be performed at a dispersion rate of between 75 pounds and 750 pounds of conditioner per 1000 ft² of the consumption apron or bedding area, as appropriate. In another example, both steps 212 and 214 are performed at a dispersion rate of between 75 pounds and 500 pounds of conditioner per 1000 ft² of the consumption apron or bedding area, as appropriate. In another example, both steps 212 and 214 are performed at a dispersion rate of between 75 pounds and 250 pounds of conditioner per 1000 ft² of the consumption apron or bedding area, as appropriate. In another example, both steps 212 and 214 are performed at a dispersion rate of between 75 pounds and 125 pounds of conditioner per 1000 ft² of the consumption apron or bedding area, as appropriate. Each of these examples is considered suitable for use in methods with ruminant animals, such as cattle, and using the acidic conditioner sodium bisulfate.

Step 214, if included, can be performed from a position within the animal containment structure and directly to bedding pack in the bedding area.

Also, step 214 can be performed while the at least one animal is positioned within the animal containment structure or while the at least one animal is temporarily outside of the animal containment structure. Advantageously, step 214 can be performed such that the conditioner is dispersed over the at least one animal while the at least one animal is in the animal containment structure.

In all methods, any suitable conditioner can be used in the dispersing step or dispersing steps included in the method. Also, in methods that include multiple dispersing steps, the conditioners used in the dispersing steps can be the same or different composition, and can include the same or different active ingredient. The inventors have determined that use of an acidic conditioner is particularly advantageous for use as the one or more conditioners dispersed in the example methods. Furthermore, the inventors have determined that sodium bisulfate is particularly advantageous for use as the active ingredient in the one or more acidic conditioners dispersed in these example methods at least because it is suitable for dispersion directly over animals contained within an animal containment structure.

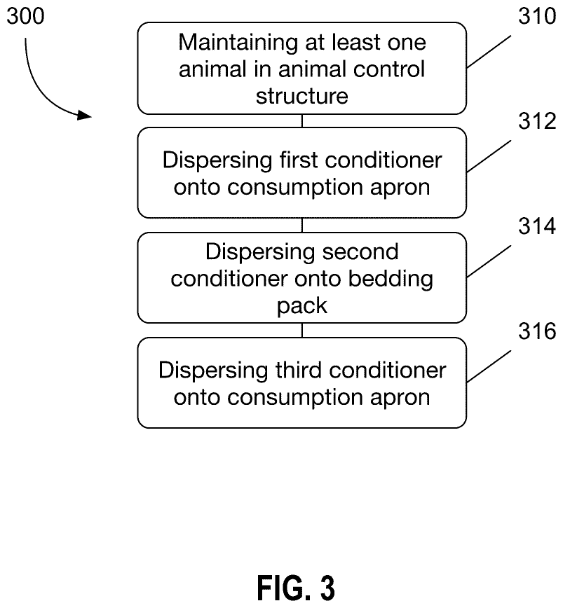

FIG. 3 is a flowchart illustration of an example method 300 of controlling ammonia in an animal containment structure having at least one consumption apron and at least one bedding area having bedding pack disposed therein. Performance of the method 300 results in controlling ammonia in the animal containment structure by lowering, at least temporarily, the pH of liquids produced during or derived from agricultural operations within the animal containment structure. For example, performance of the method 300 results in lowering, at least temporarily, the pH of a liquid obtained from the surface of the consumption apron of the animal containment structure with which the method 200 is performed and lowering, at least temporarily, the pH of a liquid solution prepared by mixing solid matter obtained from bedding pack disposed within the bedding area of the animal containment structure with a suspension fluid, such as sterile distilled water. The method 300 can be performed with any suitable animal containment structure having at least one consumption area having a consumption apron and at least one bedding area that is spatially distinct from the at least one consumption area, such as example animal containment structure 100 described above and illustrated in FIG. 1.

An initial step 310 comprises maintaining at least one animal in the animal containment structure for a maintenance period of at least two weeks. This step can be performed by conventional maintenance procedures for the relevant animal being maintained within the animal containment structure and should include at least feeding the at least one animal, cleaning both the at least one consumption apron and at least one bedding area, and replacing any bedding pack contained within the at least one bedding area. Also, this step 310 can be performed in accordance with the description of step 210 in example method 200, described above.

Another step 312 comprises dispersing a first conditioner onto the consumption apron. This step 312 can be performed at any suitable time, at any suitable frequency, any suitable number of times during the performance of step 310, and at any suitable dispersion rate, such as the dispersion rates described above for step 210 of example method 200. Also, step 312 can be performed from a position within the animal containment structure and directly to the consumption apron. Also, step 312 can be performed while the at least one animal is positioned within the animal containment structure or while the at least one animal is temporarily outside of the animal containment structure. Advantageously, step 312 can be performed such that the conditioner is dispersed over the at least one animal while the at least one animal is in the animal containment structure.

Another step 314 comprises dispersing a second conditioner onto the bedding pack. This step 314 can be performed at any suitable time, at any suitable frequency, any suitable number of times during the performance of step 310, and at any suitable dispersion rate, such as the dispersion rates described above for step 210 of example method 200. Also, step 314 can be performed from a position within the animal containment structure and directly to the bedding pack. Also, step 314 can be performed while the at least one animal is positioned within the animal containment structure or while the at least one animal is temporarily outside of the animal containment structure. Advantageously, step 314 can be performed such that the conditioner is dispersed over the at least one animal while the at least one animal is in the animal containment structure.

Another step 316 comprises dispersing a third conditioner onto the consumption apron. This step 316 is performed at least one week after performing the dispersing a first conditioner onto the consumption apron and after performing the dispersing a second conditioner onto the bedding pack. This step 316 can be performed at any suitable frequency and at any suitable dispersion rate, such as the dispersion rates described above for step 210 of example method 200. Also, step 316 can be performed from a position within the animal containment structure and directly to the consumption apron. Also, step 316 can be performed while the at least one animal is positioned within the animal containment structure or while the at least one animal is temporarily outside of the animal containment structure. Advantageously, step 316 can be performed such that the conditioner is dispersed over the at least one animal while the at least one animal is in the animal containment structure.

Advantageously, each of the first, second, and third conditioners comprises an acidic conditioner. Also advantageously, the first, second, and third conditioners comprise the same active ingredient. As described above, the active ingredient in all conditioners dispersed in performance of method 300 advantageously comprises sodium bisulfate.

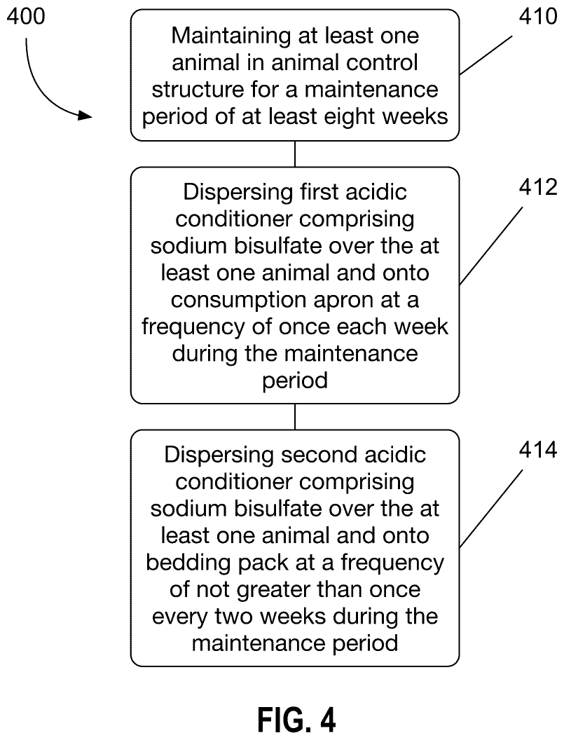

FIG. 4 is a flowchart illustration of an example method 400 of controlling ammonia in an animal containment structure having at least one consumption apron and at least one bedding area having bedding pack disposed therein. Performance of the method 400 results in controlling ammonia in the animal containment structure by lowering, at least temporarily, the pH of liquids produced during or derived from agricultural operations within the animal containment structure. For example, performance of the method 400 results in lowering, at least temporarily, the pH of a liquid obtained from the surface of the consumption apron of the animal containment structure with which the method 200 is performed and lowering, at least temporarily, the pH of a liquid solution prepared by mixing solid matter obtained from bedding pack disposed within the bedding area of the animal containment structure with a suspension fluid, such as sterile distilled water. The method 400 can be performed with any suitable animal containment structure having at least one consumption area having a consumption apron and at least one bedding area that is spatially distinct from the at least one consumption area, such as example animal containment structure 100 described above and illustrated in FIG. 1.

An initial step 410 comprises maintaining at least one animal in the animal containment structure for a maintenance period of at least eight weeks. This step can be performed by conventional maintenance procedures for the relevant animal being maintained within the animal containment structure and should include at least feeding the at least one animal, cleaning both the at least one consumption apron and at least one bedding area, and replacing any bedding pack contained within the at least one bedding area. Also, this step 410 can be performed in accordance with the description of step 210 in example method 200, described above.

Another step 412 comprises dispersing a first acidic conditioner comprising sodium bisulfate over the at least one animal and onto the consumption apron at a frequency of once each week during the maintenance period. This step 412 can be performed at any suitable dispersion rate, such as the dispersion rates described above for step 210 of example method 200. Also, step 412 can be performed from a position within the animal containment structure and directly to the consumption apron. Also, step 312 can be performed while the at least one animal is positioned within the animal containment structure or while the at least one animal is temporarily outside of the animal containment structure.

Another step 414 comprises dispersing a second acidic conditioner comprising sodium bisulfate over the at least one animal and onto the bedding pack at a frequency of not greater than once every two weeks during the maintenance period. This step 414 can be performed at any suitable dispersion rate, such as the dispersion rates described above for step 210 of example method 200. Also, step 414 can be performed from a position within the animal containment structure and directly to the bedding pack. Also, step 414 can be performed while the at least one animal is positioned within the animal containment structure or while the at least one animal is temporarily outside of the animal containment structure.

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular arrangement of elements and steps disclosed herein have been selected by the inventor simply to describe and illustrate examples of the invention and are not intended to limit the scope of the invention or its protection, which is to be given the full breadth of the appended claims and any and all equivalents thereof.

What is claimed is:

1. A method of controlling ammonia in an animal containment structure having at least one consumption area having a consumption apron and at least one bedding area that is spatially distinct from the at least one consumption area, the at least one consumption apron and at least one bedding area comprising distinct areas in the animal containment structure, the method comprising:
maintaining at least one animal in the animal containment structure for a maintenance period of at least two weeks;
dispersing a first conditioner onto the consumption apron at a frequency of once each week during the maintenance period.

2. The method of claim 1, further comprising dispersing a second conditioner onto the bedding pack at a frequency of once every two weeks during the maintenance period.

3. The method of claim 2, wherein the first conditioner comprises an acidic conditioner and the dispersing a first conditioner is performed at a dispersion rate of between 75 pounds and 750 pounds per 1000 ft² of the consumption apron.

4. The method of claim 3, wherein the second conditioner comprises an acidic conditioner and the dispersing a second conditioner is performed at a dispersion rate of between 75 pounds and 750 pounds per 1000 ft² of the bedding area.

5. The method of claim 4, wherein the dispersing a first conditioner is performed from a position within the animal containment structure and directly to the consumption apron.

6. The method of claim 5, wherein the dispersing a second conditioner is performed from a position within the animal containment structure and directly to the bedding pack.

7. The method of claim 6, wherein the dispersing a first conditioner is performed while the at least one animal is positioned within the animal containment structure.

8. The method of claim 7, wherein the dispersing a first conditioner comprises dispersing the first conditioner over the at least one animal.

9. The method of claim 8, wherein the dispersing a second conditioner is performed while the at least one animal is positioned within the animal containment structure.

10. The method of claim 9, wherein the dispersing a second conditioner comprises dispersing the second conditioner over the at least one animal.

11. The method of claim 10, wherein the first conditioner and the second conditioner comprise the same active ingredient.

12. The method of claim 11, wherein the active ingredient comprises sodium bilsulfate.

13. A method of controlling ammonia in an animal containment structure having at least one consumption area having a consumption apron and at least one bedding area that is spatially distinct from the at least one consumption area, the at least one consumption apron and at least one bedding area comprising distinct areas in the animal containment structure, the method comprising:
maintaining at least one animal in the animal containment structure for a maintenance period of at least two weeks;
dispersing a first acidic conditioner onto the consumption apron;
dispersing a second acidic conditioner onto the bedding pack;
at least one week after performing the dispersing a first acidic conditioner onto the consumption apron and after performing the dispersing a second acidic conditioner onto the bedding pack, dispersing a third acidic conditioner onto the consumption apron.

14. The method of claim 13, wherein the dispersing a first acidic conditioner is performed while the at least one animal is positioned within the animal containment structure.

15. The method of claim 14, wherein the dispersing a first acidic conditioner comprises dispersing the first acidic conditioner over the at least one animal.

16. The method of claim 15, wherein the dispersing a second acidic conditioner is performed while the at least one animal is positioned within the animal containment structure.

17. The method of claim 16, wherein the dispersing a second acidic conditioner comprises dispersing the second acidic conditioner over the at least one animal.

18. The method of claim 17, wherein the first acidic conditioner, second acidic conditioner, and third acidic conditioner comprise the same active ingredient.

19. The method of claim 18, wherein the active ingredient comprises sodium bilsulfate.

20. A method of controlling ammonia in an animal containment structure having at least one consumption area having a consumption apron and at least one bedding area that is spatially distinct from the at least one consumption area, the at least one consumption apron and at least one bedding area comprising distinct areas in the animal containment structure, the method comprising:
maintaining at least one animal in the animal containment structure for a maintenance period;
dispersing a first acidic conditioner onto the consumption apron at a first frequency during the maintenance period; and
dispersing a second acidic conditioner onto the bedding pack at a second frequency during the maintenance period, the second frequency being less than the first frequency.
